(19) United States
(12) Patent Application Publication
(10) Pub. No.: US 2015/0005484 A1
(43) Pub. Date: Jan. 1, 2015

(54) METHOD FOR SACCHARIFICATION OF BIOMASS

(71) Applicant: NIPPON SHOKUBAI CO., LTD., Osaka (JP)

(72) Inventor: Takafumi Kubo, Osaka (JP)

(21) Appl. No.: 14/374,472

(22) PCT Filed: Jan. 23, 2013
(86) PCT No.: PCT/JP2013/051247

§ 371 Date: Jul. 24, 2014

(30) Foreign Application Priority Data
Jan. 24, 2012 (JP) 2012-012388

(51) Int. Cl. C12P 19/14; C12P 19/02

(52) U.S. Cl. CPC C12P 19/14; C12P 19/02; C12P 2203/00; C12P 2201/00
USPC 536/1.11; 435/99

(57) ABSTRACT

A method for saccharification of lignocellulosic biomass, the method comprising (1) a pretreatment step of impregnating lignocellulosic biomass with an aqueous alkali solution, subjecting the resultant mixture to solid-liquid separation to remove part of the aqueous alkali solution, and then performing heat treatment, and (2) a saccharification step of enzymatically degrading the lignocellulosic biomass resulting from the pretreatment step to obtain a saccharified liquid can be applied to high-lignin lignocellulosic biomass, reduce the usage of alkali and water in the pretreatment step, increase the sugar yield in the saccharification step, decrease the reaction time, reduce enzyme adsorption on a biomass residue, and improve the enzyme recovery rate.

### TECHNICAL FIELD

The present invention relates to a method for saccharification of biomass, and more specifically to a method for enzymatic saccharification of lignocellulosic biomass.

### BACKGROUND ART

Saccharification of lignocellulosic biomass into monosaccharides for use as a fermentation feedstock is a crucial technique for utilizing inedible biomass as a resource and energy without affecting food supplies. Methods for saccharification of lignocellulosic biomass are broadly classified into acid saccharification where an acid such as sulfuric acid is used for hydrolyzation and enzymatic saccharification where an enzyme is used for hydrolyzation. Acid saccharification has an advantage in its high reaction rate, but at the same time has disadvantages such as the need for an acid-proof reactor and for a step of neutralizing and recovering acids after use. On the other hand, enzymatic saccharification allows the degradation reaction to proceed under relatively mild reaction conditions and is therefore advantageous in its low utility and equipment costs and in its high reaction selectivity as compared to acid saccharification.

To achieve a high sugar yield in enzymatic saccharification, however, a pretreatment step which facilitates enzymatic degradation of biomass is necessary, and in such a pretreatment step, higher efficiency and cost reduction are required. Enzymes account for a large proportion of the cost, and therefore reducing the cost of enzyme is practically a significant challenge.

Common pretreatment methods for enzymatic saccharification are acid pretreatment, alkali pretreatment, and hydrothermal pretreatment. The alkali pretreatment, where an alkaline compound such as NaOH is used for biomass pretreatment (Patent Literature 1 to 3), effectively degrades lignin and the like and destroys the structure of biomass so that the action of enzymes is promoted. Other advantages thereof include the relatively mild pretreatment conditions as compared to those in acid treatment and hydrothermal treatment, its applicability to high-lignin biomass that is less prone to be saccharified, and the low corrosiveness of alkali to metal. A problem in alkali pretreatment is the cost of alkali, and therefore it is needed to achieve a high sugar yield with the use of less alkali. Also needed in the pretreatment is a reduction of water usage, a further relaxation of the treatment conditions, and a reduction in the duration.

A biomass degradation product resulting from alkali pretreatment is thought to possibly interfere with fermentation and is therefore generally removed by washing with water prior to enzymatic saccharification. Hence, obtaining, with the use of less washing water, a saccharified liquid that does not interfere with fermentation is crucial in a practical use.

Meanwhile, recovering and/or reusing saccharifying enzymes is an effective measures for reducing the cost of enzyme, and various methods are known (Patent Literature 4 to 6 and Non-patent Literature 1 and 2). A saccharifying enzyme such as cellulase, however, is highly adsorptive on polysaccharides and lignin and therefore, after a saccharification reaction, is adsorbed on a hardly-degradable biomass residue. The adsorption on such a residue makes the recovery and reuse of the enzyme difficult. The degree of enzyme adsorption is known to depend on the pretreatment method employed, and the development of an effective but inexpensive pretreatment method in which the enzyme adsorption on the residue can be suppressed is desired.

Methods for the desorption and recovery of the enzyme adsorbed on the residue after the saccharification reaction are also being developed, but there is still room for improvement in the enzyme recovery rate, cost reduction, or the like.

### CITATION LIST

### Patent Literature

Patent Literature 1: JP 57-29293 A
Patent Literature 2: JP 58-98093 A
Patent Literature 3: JP 2011-83238 A
Patent Literature 4: JP 63-87994 A
Patent Literature 5: JP 2010-136702 A
Patent Literature 6: JP 2010-98951 A

### Non-Patent Literature

Non-Patent Literature 1: Biotechnology and Bioengineering, 34, 291-298 (1989)
Non-Patent Literature 2: Applied Biochemistry and Biotechnology, 143, 93-100 (2007)

### SUMMARY OF INVENTION

### Technical Problem

An object of the present invention is to provide a method for saccharification of lignocellulosic biomass, the method being applicable to high-lignin lignocellulosic biomass and capable of reducing the usage of alkali and water in a pretreatment step, increasing the sugar yield in a saccharification step, decreasing the reaction time, reducing enzyme adsorption on a biomass residue, and improving the enzyme recovery rate. Another object thereof is to provide a saccharification method for giving a saccharified liquid with excellent fermentation properties while reducing the load of removing a degradation product resulting from a pretreatment step.

### Solution to Problem

In order to solve the problems described above, the present invention provides the following.

[1] A method for saccharification of lignocellulosic biomass, the method comprising (1) a pretreatment step of impregnating lignocellulosic biomass with an aqueous alkali solution, subjecting the resultant mixture to solid-liquid separation to remove part of the aqueous alkali solution, and then performing heat treatment, and (2) a saccharification step of enzymatically degrading the lignocellulosic biomass resulting from the pretreatment step to obtain a saccharified liquid.
[2] The saccharification method according to the above [1], wherein the liquid-solid ratio of the mixture as calculated by Formula (I) is 2 to 20 before solid-liquid separation and 1 to 6 after solid-liquid separation in the pretreatment step.

Liquid-solid ratio=(total mass of all liquid components in mixture)/(mass of solid matter of lignocellulosic biomass in mixture) Formula (I)

[3] The saccharification method according to the above [1] or [2], wherein the heat treatment in the pretreatment step is performed at 100 to 200° C.
[4] The saccharification method according to any one of the above [1] to [3], wherein the saccharification step is performed in the presence of solubilized lignocellulosic biomass that is a pretreatment-degradation product resulting from the pretreatment step.
[5] The saccharification method according to any one of the above [1] to [4], the method further comprising, between the pretreatment step and the saccharification step, a removal step of partially removing a solubilized lignocellulosic biomass that is a pretreatment-degradation product resulting from the pretreatment step, wherein the content of the pretreatment-degradation product remaining in the lignocellulosic biomass after the removal step is 2 to 20% by mass as calculated by Formula (II).

Content of remaining pretreatment-degradation product=(mass of solid matter of remaining pretreatment-degradation product)/(mass of solid matter of lignocellulosic biomass) Formula (II)

[6] The saccharification method according to any one of the above [1] to [5], wherein the proportion of C5 sugar to all the sugar components in the saccharified liquid resulting from the saccharification step is 20 to 50% by mass.
[7] The saccharification method according to any one of the above [1] to [6], wherein the total sugar concentration of the saccharified liquid resulting from the saccharification step is 5 to 20% by mass.
[8] The saccharification method according to any one of the above [1] to [7], wherein an enzyme adsorbed on the lignocellulosic biomass that remains undegraded in the saccharification step is reused.
[9] The saccharification method according to any one of the above [1] to [8], wherein the heat treatment in the pretreatment step is performed with the supply of oxygen.
[10] The saccharification method according to any one of the above [1] to [9], the method further comprising, after the saccharification step, an enzyme recovery step of recovering an enzyme after the completion of the saccharification step.
[11] The saccharification method according to the above [10], wherein the enzyme recovery step includes a step of desorbing and recovering the enzyme adsorbed on the undegraded lignocellulosic biomass by alkali treatment.
[12] The saccharification method according to any one of the above [1] to [11], wherein lignocellulosic biomass with a moisture content of 30 to 90% is subjected to the pretreatment step.
[13] A saccharified liquid resulting from a saccharification step, the saccharified liquid comprising 2 to 20% by mass of solubilized lignocellulosic biomass that is a pretreatment-degradation product resulting from a pretreatment step, relative to all the sugar components in the saccharified liquid.

### Advantageous Effects of Invention

The present invention can provide a method for saccharification of lignocellulosic biomass, the method being applicable to high-lignin lignocellulosic biomass and capable of reducing the usage of alkali and water in a pretreatment step, increasing the sugar yield in a saccharification step, decreasing the reaction time, reducing enzyme adsorption after enzymatic saccharification, and improving the enzyme recovery rate. In the present invention, by reusing the recovered enzyme in a saccharification step, the usage of enzyme can be reduced and the cost of enzyme can be significantly reduced. The present invention can also give a saccharified liquid with excellent fermentation properties while reducing the load of the removal of a degradation product resulting from a pretreatment step.

### BRIEF DESCRIPTION OF DRAWINGS

FIG. 1 shows the measurements of the sugar yield and the enzyme recovery rate in Examples 1 and 2 and Comparative Example 1.

### DESCRIPTION OF EMBODIMENTS

The present invention provides a method for saccharification of lignocellulosic biomass, that is, a method for producing sugars (such as glucose, xylose, and arabinose) by saccharification of lignocellulosic biomass. The saccharification method of the present invention has only to include (1) a pretreatment step of impregnating lignocellulosic biomass with an aqueous alkali solution, subjecting the resultant mixture to solid-liquid separation so as to remove part of the aqueous alkali solution, and then performing heat treatment, and (2) a saccharification step of enzymatically degrading the lignocellulosic biomass resulting from the pretreatment step to obtain a saccharified liquid. Between the pretreatment step and the saccharification step, a removal step of partially removing solubilized lignocellulosic biomass that is a pretreatment-degradation product resulting from the pretreatment step may further be included. After the saccharification step, an enzyme recovery step of recovering an enzyme after the completion of the saccharification step may further be included.

A feedstock for the saccharification method of the present invention is not particularly limited provided that the feedstock contains lignocellulosic biomass, and may contain a high proportion of lignin. Lignocellulosic biomass (hereinafter, sometimes simply called biomass) is mainly composed of cellulose, hemicellulose, and lignin, and represents woody plants and herbaceous plants, processed materials thereof, waste materials thereof, and the like. Specific examples thereof include wood, thinnings, lumbering residues, scrap building lumber, bark, fruit bunches, fruit shells, fronds and stover, straw, bagasse, and waste paper. Preferred are palms such as oil palm, date palm, sago palm, and coconut palm (trunks, fronds, empty fruit bunches, and fruit fiber), sugarcane (bagasse and leaves), corn (cobs and stover), woods such as eucalyptus, poplar, and Japanese cedar (bark and xylem), rice straw, wheat straw, switchgrass, Napier grass, Erianthus, Miscanthus, and Miscanthus sinensis. More preferred are empty fruit bunches of palms, sugarcane bagasse, corn cobs, rice straw, wheat straw, Eucalyptus, and Japanese cedar, and further preferred is oil palm empty fruit bunch. Oil palm empty fruit bunch is biomass as a waste from palm oil extraction and available in abundance in Southeast Asia. Because of its high lignin content and high moisture content, oil palm empty fruit bunch is limited in its applications. The method of the present invention, however, is particularly effective for such a biomass feedstock. The biomass is not particularly limited in its size, shape, and the like and is preferably in the form of powder, chips, and strips obtained by shredding, pulverization, and the like and fibers obtained by fibrillation. The size of the biomass feedstock in terms of the average length of the longest side is preferably about 0.1 cm to 30 cm and further preferably about 1 cm to 10 cm. When the size falls within such a range, the biomass has excellent properties for highly successful enzymatic saccharification, solid-liquid separation, transportation, and the like. The water content (moisture content) of the biomass is not particularly limited and is preferably 0 to 90%, more preferably 30 to 90%, further preferably 40 to 80%, and particularly preferably 50 to 80% in terms of the moisture content. The lignin content, in terms of solid matter (absolute dry basis), of high-lignin lignocellulosic biomass can be 10% or more, for example, and is preferably 20% or more. The symbol “%” here and hereinafter means % by mass unless otherwise indicated.

The present invention comprises pretreatment step (1) for enhancing the efficiency of enzymatic saccharification of biomass. In the pretreatment step, biomass is impregnated with an aqueous alkali solution to give a mixture, which is then subjected to solid-liquid separation for the removal of part of the aqueous alkali solution, followed by heat treatment. This pretreatment step achieves very effective pretreatment using less alkali and less water. That is, the pretreatment facilitates the contact of an enzyme with cellulose and hemicellulose in the saccharification step and improves the efficiency of the enzymatic reaction and the sugar yield, and eventually reduces adsorption of the enzyme on a biomass residue after the saccharification reaction, leading to easy recovery of the enzyme from the residue.

A technical feature of the pretreatment method of the present invention is that impregnation of biomass with alkali is performed at a relatively high liquid-solid ratio (the ratio of liquid and solid in biomass), which is then decreased by solid-liquid separation, and heat treatment follows at the lower liquid-solid ratio. This method enables rapid and uniform impregnation of biomass with alkali and efficient action of the alkali on biomass, and can be suitably applied to high-moisture and/or high-lignin biomass.

The pretreatment step begins with the preparation of an aqueous alkali solution. Examples of an alkali compound that can be used in the aqueous alkali solution include at least one compound selected from the group consisting of hydroxides, oxides, sulfides, carbonates, and hydrogen carbonates of at least one metal selected from the group consisting of sodium, calcium, potassium, and magnesium. Ammonia can also be used. Sodium hydroxide, sodium sulfide, sodium carbonate, calcium hydroxide, potassium hydroxide, and potassium carbonate are preferred, and sodium hydroxide, calcium hydroxide, and potassium hydroxide are more preferred. The alkali compound may be used alone or as a mixture of a plurality of these. The alkali compound is dissolved in water to be used as an aqueous alkali solution. The concentration of the alkali compound in the aqueous alkali solution is preferably 0.1 to 30%, more preferably 0.5 to 20%, and particularly preferably 1 to 10%. The pH of the aqueous alkali solution is preferably 11 to 15, more preferably 12 to 14.5, and particularly preferably 12.5 to 14. To the aqueous alkali solution, an anthraquinone, such as anthraquinones and sulfonated anthraquinone, may be added. The amount of the anthraquinone to be added is not particularly limited provided that the effects of the invention are not impaired.

Subsequently, in order to prepare a mixture of the biomass and the aqueous alkali solution, that is, the biomass impregnated with the aqueous alkali solution (hereinafter, sometimes simply called a mixture), a step of bringing the aqueous alkali solution into contact with the biomass so as to impregnate the biomass with an alkali compound (impregnation step) is performed. Specifically, the biomass and the aqueous alkali solution are mixed and the resultant mixture is then subjected to treatment under various conditions so as to achieve impregnation of the biomass with the aqueous alkali solution. In the impregnation step, it is important to make the alkali reach the interior of the biomass in a rapid and uniform fashion. To achieve this, the mixture is preferably prepared so as to have a relatively high liquid-solid ratio, and specifically the liquid-solid ratio of the mixture in the impregnation step is preferably 1 to 30, more preferably 1 to 20, further preferably 2 to 20, and particularly preferably 2 to 10. The liquid-solid ratio of the mixture is calculated by the following formula.

Liquid-solid ratio=(total mass of all liquid components in mixture)/(mass of solid matter of biomass in mixture)

The expression “all (the) liquid components” refers to the sum of the liquid components in the mixture, that is, the sum of the aqueous alkali solution brought into contact with the biomass, moisture in the biomass feedstock, and all other liquids. The expression “mass of (the) solid matter of (the) biomass” refers to the mass of the solid matter of a biomass feedstock excluding liquid components such as moisture. When the liquid-solid ratio of the mixture in the impregnation step (in other words, the mixture before solid-liquid separation) falls within the above range, alkali impregnation can proceed rapidly and uniformly.

The aqueous alkali solution is brought into contact with the biomass and is then absorbed by and impregnated into the biomass. When the amount of the aqueous alkali solution (or all the liquid components) exceeds the maximum moisture content of the biomass (saturation), the aqueous alkali solution cannot completely be absorbed by the biomass and therefore present outside the biomass (in the spaces between adjacent pieces of biomass). The impregnation step is preferably performed in such conditions where the spaces are also occupied by the aqueous alkali solution.

The aqueous alkali solution brought into contact with the biomass changes its concentration when mixed with moisture and the like in the biomass, but the alkali concentration in the mixture is preferably within the range of 0.1 to 30%, more preferably within the range of 0.5 to 20%, and particularly preferably within the range of 1 to 10% in terms of the concentration of alkali compounds in all the liquid components. The overall pH of the liquid components in the mixture is preferably 11 to 15, more preferably 12 to 14.5, and particularly preferably 12.5 to 14. The overall pH of the liquid components in the mixture can be determined, for example, by diluting the mixture several-fold in water for washing, measuring the pH of the washing water, and then estimating the initial pH using the dilution factor. Preferably, the concentration and the amount of the aqueous alkali solution to be brought into contact are appropriately selected in consideration of the moisture content of the biomass, the required alkali concentration, and the like.

The treatment temperature in the impregnation step is preferably 20 to 100° C., more preferably 20 to 70° C., and particularly preferably 20 to 50° C. The impregnation step may be performed at normal pressure, or under reduced or increased pressure and such pressure control can accelerate the alkali impregnation. The pressure, when applied, is preferably 0.01 to 2 MPaG (gauge pressure) and more preferably 0.05 to 0.5 MPaG. The duration of impregnation is preferably 0.1 to 10 hours, more preferably 0.1 to 3 hours, and particularly preferably 0.1 to 1 hour. The impregnation step can be performed either batch-wise or continuously, and may be performed either in a stationary manner or with mixing, stirring, liquid circulation, or the like for enhanced rate of impregnation.

After the preparation of the mixture, i.e., impregnation of the lignocellulosic biomass with the aqueous alkali solution, solid-liquid separation is carried out so as to remove part of the aqueous alkali solution. The expression “part of the aqueous alkali solution” refers to the part removable by solid-liquid separation from the mixture prepared in the impregnation step. In the mixture, the aqueous alkali solution is assumed to be present inside and outside (that is, in the spaces between) pieces of the biomass. The part of the aqueous alkali solution that is removed by solid-liquid separation is principally the aqueous alkali solution present in the spaces between pieces of the biomass but may include the aqueous alkali solution inside the biomass depending on the conditions of solid-liquid separation. The purpose of carrying out solid-liquid separation is to remove principally the portion of the aqueous alkali solution in the space between pieces of the biomass so as to reduce the liquid-solid ratio. Reducing the liquid-solid ratio will limit the reaction field and thereby dramatically enhance the efficiency of alkali to act on the solid biomass.

Solid-liquid separation can be carried out, for example, by filtration, centrifugation, or centrifugal filtration, or with a cyclone, a filter press, a screw press, or a decanter. The mixture obtained after the solid-liquid separation (hereinafter, sometimes called alkali-impregnated biomass) is then subjected to subsequent heat treatment step. The part of the aqueous alkali solution that has been removed by the solid-liquid separation is preferably reused in an impregnation step. The part of the aqueous alkali solution that has been removed by the solid-liquid separation contains a trace amount of a biomass-derived component and therefore, when reused, can likely be effective in improving the rate of alkali impregnation, decreasing alkali usage, and the like. The loss of the alkali compound or the aqueous alkali solution may be compensated for, where appropriate, prior to reuse.

As for the alkali-impregnated biomass (in other words, the mixture after the solid-liquid separation), the liquid-solid ratio (=(total mass of all liquid components in mixture)/(mass of solid matter of lignocellulosic biomass in mixture)) is preferably 1 to 10, more preferably 1 to 6, and particularly preferably 1 to 4. In the mixture after the solid-liquid separation, the amount of the alkali compound contained in all the liquid components (alkali impregnation ratio) relative to the mass of the solid matter of the biomass is preferably 0.1 to 30%, more preferably 1 to 20%, and particularly preferably 2 to 15%.

Subsequently, the alkali-impregnated biomass is subjected to heat treatment. The temperature during the heat treatment is preferably 20 to 250° C., more preferably 100 to 200° C., and particularly preferably 150 to 200° C. The duration of heat treatment is preferably 0.1 to 100 hours, more preferably 0.1 to 24 hours, and particularly preferably 0.1 to 1 hour. Heat treatment within the ranges of temperature and duration increases the sugar yield and the enzyme recovery rate.

The atmosphere in the gas phase during the heat treatment is not particularly limited and is oxygen gas, nitrogen gas, an oxygen/nitrogen mixed gas, air, or the like. Alkali pretreatment performed in the presence of oxygen results in less enzymes to be adsorbed on the biomass in the saccharification step, and therefore likely increases the sugar yield and the enzyme recovery rate. Specifically, the oxygen concentration is preferably 1 to 100% by volume, more preferably 10 to 95% by volume, and particularly preferably 15 to 80% by volume. In a preferred embodiment, air, which is inexpensive, is used. In the case of heat treatment in the presence of oxygen, since oxygen is consumed over time, heat treatment is preferably performed with the supply of oxygen (so as to maintain the oxygen concentration).

The pressure (gauge pressure) during the heat treatment is not particularly limited, and is preferably 5 MPaG or lower, more preferably 3 MPaG or lower, and particularly preferably 1 MPaG or lower. In the present invention that includes removing the aqueous alkali solution in the spaces between pieces of the biomass, the surface area of the biomass is large enough to efficiently take in the gas from the gas phase. Therefore, pretreatment in the presence of oxygen is preferred in the present invention.

By the heat treatment, the biomass (mostly lignin that is contained therein) is degraded and solubilized to give a compound having a phenolic hydroxy group and/or a carboxy group. This degradation consumes (neutralizes) an alkali component, and therefore the pH of the biomass decreases. The overall pH of the liquid components in the mixture after the heat treatment is preferably 6 to 14, more preferably 7 to 13, and particularly preferably 8 to 12. When the pH falls within such a range, the alkali is efficiently used and the load of the subsequent removal step can be reduced. The overall pH of the liquid components after the heat treatment can be estimated by the method described above.

The heat treatment conditions such as temperature, atmosphere in the gas phase, and pressure may be changed during the course of the heat treatment. For example, as the pretreatment, heat treatment without the supply of oxygen (or with a limited amount of oxygen) and heat treatment with the supply of oxygen may be sequentially carried out. The treatment conditions in each of these phases of heat treatment are the same as above and can be combined as needed.

The heat-treated biomass may be subjected as it is to the subsequent saccharification step, but preferably subjected to, prior to the saccharification step, a step (a removal step) of removing part of the solubilized biomass degradation product produced in the pretreatment step (hereinafter, sometimes called the pretreatment-degradation product) (hereinafter, biomass after the removal step is sometimes called pretreated biomass). The pretreatment-degradation product refers to soluble solid matter resulting from alkali degradation of the biomass. The degradation product consists of multiple components such as degraded lignin as the main component as well as an organic acid (such as acetic acid) and also contains an alkali component. The removal of the pretreatment-degradation product is performed by washing the biomass with a washing solvent such as water or by solid-liquid separation by pressing, centrifugation, or the like, and preferably by washing with water. In the removal by washing with water, another solvent, for example, may be added to and mixed with the washing water. Specifically, an organic solvent such as alcohols and ketones or an acid for pH adjustment may be added. The amount of the washing solvent containing water is preferably 0.1 to 100 times, more preferably 0.5 to 30 times, and particularly preferably 1 to 10 times the mass of the heat-treated, alkali-impregnated biomass. The washing solvent is added to the heat-treated biomass so as to elute the pretreatment-degradation product, and then solid-liquid separation is performed to separate the pretreated biomass from the washing medium (containing the pretreatment-degradation product). Washing can be performed under the same conditions as in the impregnation step, and may be performed once or multiple times. Washing can be performed batch-wise, semi-batch-wise, or continuously, but semi-batch-wise or continuous washing is preferred for high efficiency. Washing may be followed by drying, which, however, may tighten the structure of the biomass. Therefore, the biomass containing water is preferably subjected to the subsequent saccharification step without drying.

Removal by solid-liquid separation by means of pressing, centrifugation, or the like is advantageous because the usage of water can be decreased. Subjecting the heat-treated biomass to pressing or centrifugation so as to remove the liquid component contained in the biomass can also remove part of the pretreatment-degradation product. Washing with water and solid-liquid separation by pressing, centrifugation, or the like may be carried out in combination.

Since the pretreatment-degradation product, when present at a high concentration in the saccharified liquid, can adversely affect fermentation, part of the pretreatment-degradation product is preferably removed in the removal step. On the other hand, the present inventors found that the presence of the pretreatment-degradation product reduces non-specific enzyme adsorption on the biomass. The inventors also revealed that the presence of the pretreatment-degradation product in the saccharification step and/or the enzyme recovery step offers advantages of increasing the sugar yield, decreasing the enzyme usage, improving the enzyme recovery rate, and the like. In addition, the inventors found that the presence of the pretreatment-degradation product during fermentation at an amount within a certain range does not interfere with but instead favorably affect fermentation (by increasing the production and/or improving the fermentation rate, for example). Therefore, the conditions in the removal step are preferably selected so as not to completely remove but instead to leave part of the pretreatment-degradation product on purpose. In this case, the content of the pretreatment-degradation product remaining in pretreated biomass is preferably 1 to 30%, more preferably 2 to 20%, and particularly preferably 5 to 20%. The content of the remaining pretreatment-degradation product is calculated by the following formula.

Content of remaining pretreatment-degradation product=(mass of solid matter of remaining pretreatment-degradation product)/(mass of solid matter of lignocellulosic biomass)

The mass of the solid matter of the remaining pretreatment-degradation product can be determined by sampling the pretreated biomass, washing the sample thoroughly (for removing the pretreatment-degradation product adequately), and then measuring the amount of the solid matter (in other words, the amount of the pretreatment-degradation product) contained in the washing medium. The mass of the solid matter of the lignocellulosic biomass is the mass of the solid matter of the pretreated biomass not containing pretreatment-degradation product, that is, the mass of the solid matter of the pretreated biomass after thoroughly washed.

The pretreatment-degradation product within the above range favorably affects the saccharification step, the enzyme recovery step, or fermentation, and gives advantages such as an increased sugar yield, an improved enzyme recovery rate, and an increased fermentation yield. The acceptable content of the remaining pretreatment-degradation product is relatively high, which is advantageous in reducing the load of the removal step, decreasing the usage of washing water, and the like.

In the subsequent saccharification step (2), the pretreated biomass resulting from the pretreatment step (1) is enzymatically degraded to give a saccharified liquid. That is, an enzyme and, where appropriate, water and a pH-adjusting agent are added to the pretreated biomass to give a mixture (hereinafter, sometimes called a reaction mixture) for the following saccharification reaction. Water and/or a pH-adjusting agent, when added, may be added concurrently with or separately from the enzyme. Alternatively, a pH-adjusting agent may be added in the removal step so as to adjust the pH in advance. Addition of the enzyme is preferably performed after pH adjustment. The enzyme has only to contain an enzyme capable of hydrolyzing cellulose into a monosaccharide (glucose) or an enzyme capable of hydrolyzing hemicellulose into monosaccharides (such as xylose, mannose, and arabinose). Such an enzyme, which is generally called cellulase or hemicellulase, is composed of a plurality of enzymes. The enzyme used in the saccharification method of the present invention has only to contain a cellulase or a hemicellulase but preferably contains both for improving saccharification efficiency. In the pretreatment of the present invention, where alkali pretreatment efficiently proceeds, solubilization of hemicellulose tends not to proceed readily and the pretreated biomass has a high hemicellulose content. For this reason, a method comprising the use of an enzyme composition containing both cellulase(s) and hemicellulase(s) so as to degrade cellulose and hemicellulose simultaneously is a preferred embodiment. Such simultaneous enzymatic degradation offers advantages such as a reduced reaction time and an increased sugar concentration. In a pretreatment method that solubilizes hemicellulose (namely, acid treatment, hydrothermal treatment, alkali treatment under stringent conditions, or the like), it is necessary to degrade cellulose and hemicellulose separately.

A preferred cellulase contains cellobiohydrolase, β-glucanase, and β-glucosidase. A preferred hemicellulase contains xylanase and β-xylosidase. Examples of other hemicellulases include acetylxylan esterase, α-arabinofuranosidase, mannanase, α-galactosidase, xyloglucanase, pectolyase, and pectinase. Other enzymes involved with degradation of plant cell wall, such as ferulic acid esterases, coumaric acid esterases, and proteases, may also be contained. Whether each of these enzymes is contained can be determined by evaluating the enzyme activity using a substrate for the enzyme.

The origin of the enzyme is not particularly limited, and enzymes derived from microorganisms of the genus Trichoderma, the genus Acremonium, the genus Aspergillus, the genus Phanerochaete, the genus Humicola, the genus Bacillus, and the like are exemplified. Preferred enzymes are ones derived from the genus Trichoderma, the genus Acremonium, and the genus Aspergillus, and further preferred are ones derived from the genus Trichoderma.

These enzymes are commercially available and can be suitably used in the method of the present invention. Examples of commercially available enzyme preparations (trade name) include Cellic products (such as CTec and HTec), Novozyme 188, Celluclast, and Pulpzyme manufactured by Novozymes, Accellerase products (such as TRIO and DUET) and Multifect products manufactured by Genencor, Meicelase manufactured by Meiji Seika Kaisha Ltd., Onozuka manufactured by Yakult, and Cellulase (A and T) manufactured by Amano Enzyme Inc. Cellic products and Accellerase products are preferred. These enzyme preparations contain cellobiohydrolase, β-glucanase, β-glucosidase, xylanase, and β-xylosidase and can be used alone or as a combination of a plurality of these considering the composition of the biomass feedstock and the activity of the enzymes contained. It is preferred to use an enzyme preparation with high cellulase activity and an enzyme preparation with high hemicellulase activity in combination. For example, Cellic CTec products (predominantly composed of cellulases) and Cellic HTec products (predominantly composed of hemicellulases) are preferably used in combination.

In the present invention, the enzyme adsorbed on an undegraded biomass residue is preferably recovered by alkali treatment for reuse. When reuse is taken into account, a preferred enzyme to use is one having high alkaline stability and high thermal stability. The enzyme may be modified chemically or genetically (by protein engineering). Such modification can enhance the stability, reduce the adsorptivity on a residue, and enhance the efficiency of recovery, and therefore can be suitably employed in the present invention.

The amount of the enzyme to be used is not particularly limited, and is preferably 0.01 to 10% and more preferably 0.05 to 5% in terms of the mass of the solid matter of the component having enzyme activity (or the mass of the proteins) relative to the mass of the solid matter of the pretreated biomass. The amount of water to be added is not particularly limited; in fact, there is no need to add water when the pretreated biomass contains enough water. The amount of water is preferably 0 to 20 times and further preferably 0 to 10 times the mass of the solid matter of the pretreated biomass. The solid content of the pretreated biomass in the reaction mixture is preferably 1 to 50%, more preferably 3 to 30%, and particularly preferably 5 to 25%.

As the pH-adjusting agent, a suitably selected acid or alkali can be used as needed. When alkali treatment is performed in the pretreatment step, the pretreated biomass is alkaline and therefore an acid is used to adjust the pH to the range suitable for saccharification. The acid here is not particularly limited, and examples thereof include sulfuric acid, hydrochloric acid, nitric acid, phosphoric acid, acetic acid, citric acid, succinic acid, and carbon dioxide, among which sulfuric acid, hydrochloric acid, acetic acid, and carbon dioxide are preferred. The reaction conditions in the saccharification step are not particularly limited provided that they allow enzymatic hydrolyzation to proceed. The reaction temperature is usually 20 to 80° C., preferably 30 to 60° C., and more preferably 40 to 55° C. The reaction time is usually 1 to 300 hours, preferably 10 to 150 hours, and more preferably 20 to 100 hours. The pH during the reaction may be set based on the optimum pH of the enzyme and is usually 3 to 7, preferably 4 to 6, and more preferably 4.5 to 5.5. For pH adjustment, an additional amount of the pH-adjusting agent described above and/or a buffer component may be used. Specific examples of the buffer component that can be used include various organic acids, and preferred are acetic acid, citric acid, and succinic acid.

For enhancing the action of the enzyme, the saccharification step may be performed in the presence of various compounds, and examples of the compounds include proteins, surfactants, and lignin degradation products. These compounds have an effect of reducing non-specific adsorption of the enzyme on the biomass, and therefore can offer advantages of improving the rate of saccharification reaction and the enzyme recovery rate, decreasing the usage of the enzyme, and the like. The lignin degradation products are preferred, and the pretreatment-degradation product resulting from the pretreatment step is more preferred. The pretreatment-degradation product is inexpensive because it is a by-product of the process, and is also highly effective. Therefore, a method comprising the saccharification step performed in the presence of the pretreatment-degradation product is a preferred embodiment of the present invention. The presence of the pretreatment-degradation product in the saccharification step may be ensured by using the pretreated biomass prepared as described above (in which the pretreatment-degradation product remains), or by adding the pretreatment-degradation product separately. The concentration of the pretreatment-degradation product is preferably 1 to 30%, more preferably 2 to 20%, and particularly preferably 5 to 20% relative to the mass of the solid matter of the pretreated biomass. The method for the saccharification reaction is not particularly limited, and the saccharification reaction may be performed with stirring or liquid circulation or in a stationary manner. In order to facilitate the saccharification reaction, stirring or liquid circulation is preferably performed.

The saccharification step gives a reaction mixture that contains a saccharified liquid. The reaction mixture is a mixture of the saccharified liquid (liquid containing soluble, low-molecular sugars resulting from hydrolysis of the biomass, and free enzymes) and a residue (undegraded biomass, which is a solid having enzymes adsorbed thereon). The saccharified liquid may be utilized as it is in the form of the reaction mixture (that is, as a mixture with the residue) or utilized after separated from the residue by solid-liquid separation or the like (this process is regarded as part of the enzyme recovery step described later). The residue has saccharifying enzymes adsorbed thereon, and reusing the enzyme adsorbed on the residue is a preferred embodiment of the present invention. Reusing the adsorbed enzyme can decrease the usage of enzyme. Examples of the method for reusing the adsorbed enzyme include (a) a method of directly reusing the undegraded biomass having the adsorbed enzyme in a reaction and (b) a method of desorbing and recovering the adsorbed enzyme from the residue for reuse.

Specific examples of method (a) include separating the undegraded biomass (having the enzyme adsorbed thereon) from the saccharified liquid during or after the saccharification reaction and then using the undegraded biomass in the subsequent round of saccharification reaction. Preferably, recovery of the saccharified liquid (separation from the undegraded biomass) and addition of a fresh biomass feedstock are performed sequentially or continuously during the saccharification reaction, and the adsorbed enzyme is continuously used in the course of the saccharification reaction. A specific method for (b) will be described later.

Carrying out the enzyme recovery step after the completion of the saccharification step is a preferred embodiment of the present invention. Enzyme adsorption on biomass is a problem in enzyme recovery, but the pretreatment method of the present invention can reduce the enzyme adsorption and therefore allow more efficient enzyme recovery.

As described above, in the enzyme recovery step, the reaction mixture resulting from the saccharification step is subjected to solid-liquid separation for separation of the saccharified liquid from the residue. The method for solid-liquid separation is not particularly limited, and filtration, centrifugation, centrifugal filtration, or the use of a cyclone, a filter press, a screw press, or a decanter can be employed, for example. From the saccharified liquid, free enzymes (non-adsorbed enzyme) can be recovered, while from the residue, adsorbed enzymes can be recovered. As described above, the recovered enzyme may be reused as it is or be desorbed and recovered from the residue prior to reuse.

The method for recovering the enzyme from the residue is not particularly limited, and examples thereof include washing the residue with water, using an acid for enzyme recovery, and using alkali for enzyme recovery (alkali treatment). A method including alkali treatment is preferred because alkali treatment desorbs the adsorbed enzyme and achieves a high enzyme recovery rate.

The method for the alkali treatment is not particularly limited provided that it includes adding alkali to act on the residue. The alkali treatment may be performed either before or after solid-liquid separation of the reaction mixture. A preferred method includes (A) a step of performing alkali treatment by adding alkali to the reaction mixture before solid-liquid separation, (B) a step of adding alkali (and water) to the residue left after the solid-liquid separation for alkali treatment, or a combination of these steps. In any step, it is important to adjust the pH of the alkali treatment liquid (the entire treatment liquid containing the residue, alkali, and water, or the entire reaction mixture) to a predetermined range. This is because enzyme adsorption on or enzyme desorption from the residue depends on the pH of the treatment liquid. While more enzyme desorbs with increase in the pH, a problem of enzyme inactivation due to the alkali occurs at high pH. The pH of the alkali treatment liquid after alkali addition is preferably 6 to 11, more preferably 7 to 10, and particularly preferably 7.5 to 9.5.

The alkali compound to use in the alkali treatment can be the same as the alkali compounds exemplified for the pretreatment step, and preferred compounds to use in the alkali treatment are also the same as those preferred for the pretreatment step.

The alkali compound is preferably added as an aqueous solution. The concentration and the amount of the alkali compound to be added are not particularly limited as long as they are appropriate to achieve the pH range. However, a too low concentration increases the addition amount, resulting in a decrease in the concentration of the recovered enzyme. Therefore, the alkali concentration of the aqueous alkali solution to be added is preferably 0.01 to 10% and is more preferably 0.1 to 5%.

During the alkali treatment, an additive may be supplied so as to facilitate enzyme desorption. Examples of the additive include proteins, surfactants, and lignin degradation products, and preferred among these are lignin degradation products. Adding the pretreatment-degradation product resulting from the pretreatment step during the alkali treatment is a preferred embodiment of the present invention, where the amount of the pretreatment-degradation product to be added is 1 to 30%, more preferably 2 to 20%, and particularly preferably 5 to 20% relative to the mass of the solid matter of the residue.

During the alkali treatment, stirring, heating, and/or the like may be performed so as to facilitate enzyme desorption from the residue. The temperature in the alkali treatment is preferably 5 to 60° C. and is more preferably 10 to 40° C. The duration of the treatment is preferably 0.1 to 10 hours and is more preferably 0.1 to 1 hour.

Following the alkali treatment, solid-liquid separation is carried out so as to separate the alkali treatment liquid (enzyme recovery liquid) from the residue. The method for solid-liquid separation here is not particularly limited, and filtration, centrifugation, centrifugal filtration, or the use of a cyclone, a filter press, or a decanter can be employed, for example.

The process from the alkali treatment to solid-liquid separation may be carried out either batch-wise or continuously. Step (A) is preferably performed batch-wise. Step (B) may be performed either batch-wise or continuously but is preferably performed continuously.

When step (B) is performed batch-wise, alkali treatment (and the recovery of the alkali treatment liquid) may be carried out once or multiple times. When performed multiple times, the pH of the alkali treatment liquid is preferably progressively increased (gradually raised). Even when step (B) is performed continuously, the pH of the alkali treatment liquid is preferably progressively increased. With the pH being progressively increased during the alkali treatment, the enzyme recovery proceeds more mildly and more efficiently. In the alkali treatment where the pH is progressively increased, the pH of the alkali treatment liquid (enzyme recovery liquid) at the end point of the progressive increase is preferably 6 to 11, more preferably 7 to 10, and particularly preferably 7.5 to 9.5.

A saccharifying enzyme is composed of a plurality of enzymes, and it has been confirmed that each of the enzymes has different adsorption/desorption properties. Even when a saccharifying enzyme used is a mixture of a plurality of enzymes that are different in the pH suitable for desorption and in stability (for example, a mixture of a plurality of cellulases and a plurality of hemicellulases), the alkali treatment step (B) performed with the pH of the alkali treatment liquid progressively increased gives a high enzyme recovery rate and is therefore very useful.

To the enzyme recovery liquid resulting from the enzyme recovery step, an acid is preferably added immediately after the recovery so as to adjust the pH to weakly acidic to neutral. The pH of the enzyme recovery liquid after the addition of an acid is preferably 3 to 7 and is further preferably 4 to 6.

The enzyme recovery liquid can be reused in a saccharification step. Prior to reuse, the enzyme recovery liquid is concentrated by ultrafiltration or the like as needed. Since the saccharified liquid resulting from solid-liquid separation of the reaction mixture also contains free enzymes (and sugars), the liquid is preferably subjected to ultrafiltration or the like so that the enzyme separated from the sugar can be reused. Alternatively, by taking advantage of the characteristic adsorption thereof on a biomass feedstock, the enzyme can be easily reused. That is, by bringing the saccharified liquid or the enzyme recovery liquid into contact with a fresh biomass feedstock, which then adsorbs the enzyme alone, the sugar and the enzyme (adsorbed on the biomass feedstock) can be separated by solid-liquid separation. When the sugar is utilized as a fermentation feedstock, the enzyme recovery liquid containing the sugar may be subjected to fermentation as it is. When the enzyme recovery liquid is reused in a saccharification reaction, a fresh enzyme may be added. The composition of this additional enzyme may be the same as that of the initial enzyme. However, since the composition of the recovered enzyme may have changed, the additional enzyme is preferably selected, as appropriate, depending on the enzyme activity after recovery. For example, β-glucosidase is readily adsorbed on a reaction residue and sometimes the recovery rate is lower as compared to other enzymes, and therefore in this case, it is preferred to add an enzyme liquid containing β-glucosidase in abundance.

The product obtained in the present invention is low-molecular sugars, the pretreatment-degradation product, and the undegraded residue. Examples of the sugar include monosaccharides, disaccharides, and oligosaccharides. Specific examples thereof include glucose, mannose, galactose, xylose, arabinose, glucuronic acid, galacturonic acid, cellobiose, xylobiose, cellooligosaccharide, xylooligosaccharide, and the like. Before use, the disaccharides and the oligosaccharides may be converted into monosaccharides with the use of an enzyme or the like.

The application of the resultant sugar is not particularly limited and such a sugar can be suitably used as a fermentation feedstock, a chemicals feedstock, feed, fertilizer, or the like. As a fermentation feedstock, the sugar can be suitably used in fermentative production of chemicals such as ethanol, 1-butanol, isobutanol, 2-propanol, lactic acid, succinic acid, acetic acid, 3-hydroxypropionic acid, pyruvic acid, citric acid, acrylic acid, itaconic acid, fumaric acid, various amino acids, isoprene, and 1,3-propanediol. When such a sugar is used in fermentation, the Saccharification step and the fermentation step may be performed either separately or simultaneously. Alternatively, it is allowable that the saccharification step alone is performed partway, followed by simultaneous saccharification and fermentation. The pretreatment-degradation product resulting from the pretreatment step contains a lignin degradation product resulting from alkali degradation of biomass, and can be used as an additive in the saccharification step and/or the like. The lignin degradation product can be used as a chemical. Since the washing medium and/or the like resulting from the removal step contains alkali for use in the pretreatment step, the alkali may be recovered and reused. The recovery and the reuse of the alkali can be performed, for example, by a method that is generally known for use in the pulp production process (such as black liquor evaporation, combustion, dissolution of combustion residues, and causticization). The residue can be used as a biofuel in the production of steam and electric power.

The apparatus for use in each step is not particularly limited. A reactor that can be used in the pretreatment step and the saccharification step is a batch reactor, a continuous reactor, or a semi-continuous reactor, for example. Specific examples thereof include a batch reaction tank equipped with a filter (a strainer), a continuous reactor equipped with a screw feeder, a semi-continuous reaction tank for continuous or sequential operation of adding a biomass feedstock and extracting the reaction liquid therefrom, and a column-type, filling-up reaction tank. Preferably used in the pretreatment step is a continuous reactor equipped with a screw feeder. In this case, part of the aqueous alkali solution is removed by solid-liquid separation at the entrance of the screw feeder, and charging is performed while the pressure is raised. The saccharification step is preferably performed as follows: after a reactor is filled with a biomass feedstock, a saccharification reaction is allowed to proceed while solid-liquid separation and circulation of the saccharified liquid are carried out. Alternatively, the pretreatment step, the removal step, the saccharification step, and the enzyme recovery step can be carried out in the same reactor (one-pot reaction). The apparatus for solid-liquid separation can be a filter press, a screw press, a centrifuge, a centrifugal filter, a cyclone, a decanter, or the like. In the enzyme recovery step, the same apparatus (reactor) as in the saccharification step can be used, and preferably the apparatus allows continuous operation of adding the aqueous alkali solution and extracting the enzyme recovery liquid. It is also allowable to use the apparatus used in the saccharification step as it is. Various kinds of apparatus used in pulp production can be used in the saccharification method of the present invention. In the pretreatment step of the present invention, a continuous digester known as Kamyr-type or the like can be used, for example. Alternatively, the pretreatment step can be carried out with oxygen being supplied with the use of an oxygen-based bleaching tower.

The recovery rate as for the enzyme resulting from the saccharification method of the present invention (the enzyme activity of the recovered enzyme relative to the enzyme activity of the enzyme used in the saccharification step) is very high, and therefore the recovered enzyme can be efficiently reused. According to the saccharification method of the present invention, the total of the enzyme recoverable from the residue and the enzyme recoverable from the saccharified liquid is at least 50% or more, and conditionally 70% or more, in terms of enzyme activity relative to the enzyme used in the saccharification step. Thus, the saccharification method of the present invention can decrease the usage of enzyme and significantly reduce the cost of enzyme, and is therefore a very useful approach.

The sugar yield of the lignocellulosic biomass according to the present invention is not particularly limited and is preferably 65% or more, more preferably 75% or more, and particularly preferably 85% or more in terms of glucose yield (%) calculated by the following formula.

Glucose yield %=(amount of glucose produced)/(theoretical glucose amount obtainable from biomass feedstock(solid content basis))

The sugar yield of the lignocellulosic biomass according to the present invention is preferably 80% or more in terms of C5-sugar yield (%) calculated by the formula below. The C5 sugar refers to xylose, arabinose, xylobiose, and/or the like.

C5-Sugar yield %=(total amount of C5 sugars produced)/(theoretical total C5−sugar amount obtainable from biomass feedstock(solid content basis))

The sugar yield of the lignocellulosic biomass according to the present invention is preferably 70% or more, more preferably 75% or more, and particularly preferably 80% or more in terms of the total yield of sugars including glucose and C5 sugars.

The proportion of C5 sugars to all the sugar components in the saccharified liquid resulting from the saccharification step is preferably 20 to 50%, more preferably 25 to 45%, and particularly preferably 30 to 45%. The “C5 sugars” refers to the same as above, and “all the sugar components” means the sum of the sugar components including C5 sugars and C6 sugars (such as glucose). In spite of the fact that a C5 sugar degrades more readily than a C6 sugar does and is therefore less prone to give a high yield, the method of the present invention characteristically gives a high C5-sugar yield and hence can increase the proportion of C5 sugars and the like to the above range. When the proportion of C5 sugars falls within the above range, advantages such as improved efficiency of C5 sugar use in fermentation are obtained.

The total sugar concentration of the saccharified liquid resulting from the saccharification step is preferably 5 to 20% and is more preferably 7 to 15%. The total sugar concentration refers to the total concentration of all the sugar components in the saccharified liquid. When the total sugar concentration is within the above range, the efficiency of sugar use in fermentation is improved and the saccharification step can be performed efficiently.

The present invention also encompasses a saccharified liquid characterized by comprising a certain concentration of the pretreatment-degradation product resulting from the pretreatment step. The concentration of the pretreatment-degradation product in the saccharified liquid is preferably 1 to 30%, more preferably 2 to 20%, and particularly preferably 5 to 20% relative to all the sugar components in the saccharified liquid. The pretreatment-degradation product at a concentration within the above range does not interfere with fermentation but have advantages in increasing the fermentation product, enhancing the fermentation rate, and the like, and is also advantageous in the preparation of the saccharified liquid because of its effects such as a reduction in the load of the removal step of removing the pretreatment-degradation product, a decrease in the usage of washing water, improvement in the reaction efficiency in the saccharification step, and a decrease in the usage of enzyme. Examples of the method for ensuring the presence of the pretreatment-degradation product in the saccharified liquid include leaving the pretreatment-degradation product in the pretreated biomass and then performing saccharification, adding the pretreatment-degradation product during the saccharification step, and adding the pretreatment-degradation product to the resultant saccharified liquid. The concentration of the pretreatment-degradation product in the saccharified liquid can be determined by analyzing the saccharified liquid (by chromatography or the like) and quantifying the lignin degradation product (phenolic polymers or single-molecule compounds) and the like. In the quantification, an isolated pretreatment-degradation product is preferably used as an authentic sample. By quantifying all the sugar components in the saccharified liquid, the concentration of the pretreatment-degradation product relative to all the sugar components can also be determined.

The enzyme recovery rate in the present invention is not particularly limited. The recovery rate as for cellobiohydrolase (CBH) is preferably 40% or more, more preferably 55% or more, and particularly preferably 60% or more; the recovery rate as for β-glucosidase (GLD) is preferably 10% or more, preferably 30% or more, and preferably 50% or more; the recovery rate as for β-xylosidase (XLD) is preferably 30% or more, more preferably 40% or more, and particularly preferably 50% or more; the recovery rate as for carboxymethyl cellulase (CMC) is preferably 40% or more, more preferably 45% or more, and particularly preferably 50% or more; and the recovery rate as for xylanase (XYN) is preferably 40% or more, more preferably 45% or more, and particularly preferably 50% or more. Particularly preferably, all of these requirements on enzyme recovery rate are satisfied.

### EXAMPLES

The present invention will be described in more detail by examples. The scope of the present invention is, however, not limited to these examples, and various modifications can be made by a person with ordinary skill in the art without departing from the technical spirit of the present invention. In the examples below, CBH denotes cellobiohydrolase, GLD denotes β-glucosidase, XLD denotes β-xylosidase, CMC denotes carboxymethyl cellulase (β-glucanase), and XYN denotes xylanase.

### [Material Used in Experiment]

### (1) Biomass

As a lignocellulosic biomass feedstock, oil palm empty fruit bunch (hereinafter, called “EFB”) (of Indonesian origin) generated from palm oil production was used. The EFB was shredded fiber obtained from a palm oil mill.

### (2) Saccharifying Enzyme

Enzyme liquids Cellic CTec2 (trade name, hereinafter called “enzyme A”) and Cellic HTec2 (trade name, hereinafter called “enzyme B”) both from Novozymes were mixed at a predetermined proportion. The enzyme activity of enzyme A was mainly attributed to cellulases (CBH, GLD, CMC) while that of enzyme B was mainly attributed to hemicellulases (XLD, XYN).

### [Method of Analysis]

Enzyme activity was measured by the methods below. Specifically, measurement methods disclosed in JP 2012-223113 A were employed.

CBH activity: colorimetric assay using p-nitrophenyl-β-D-cellobioside as a substrate.
GLD activity: colorimetric assay using p-nitrophenyl-β-D-glucopyranoside as a substrate.
XLD activity: colorimetric assay using p-nitrophenyl-β-D-xylopyranoside as a substrate.
CMC activity: colorimetric assay using carboxymethylcellulose as a substrate. Reducing sugars were quantified by the DNS (dinitrosalicylic acid) assay.
XYN activity: colorimetric assay using soluble xylan as a substrate. Reducing sugars were quantified by the DNS assay.

After saccharification, generated sugars were quantified by HPLC (high-performance liquid chromatography). The column used was Shodex (registered trademark) Sugar KS-801 (trade name, column for ligand exchange chromatography, particle diameter: 6 μm, maximum usable pressure: 5.0 MPa, working flow rate: 0.5 to 1.0 mL/min, manufactured by Showa Denko K.K.). A differential refractometer (RI) was used for detection. In analysis, deionized water was used as the mobile phase and the temperature of the column was 60° C.

A sugar yield and an enzyme recovery rate were calculated as follows.

Glucose yield %=(amount of glucose produced)/(theoretical glucose amount obtainable from biomass feedstock(untreated,solid content basis))C5-sugar yield %=(total amount of C5 sugars produced)/(theoretical total C5−sugar amount obtainable from biomass feedstock(untreated,solid content basis))

Total sugar yield %=(amount of glucose produced+total amount of C5 sugars produced)/(theoretical total sugar amount obtainable from biomass feedstock(untreated,solid content basis))

The “C5 sugars” here refers to xylose, arabinose, and xylobiose. The theoretical sugar yield from oil palm empty fruit bunch (EFB) feedstock was 42% for glucose, 26% for the sum of C5 sugars (25% for xylose and 1% for arabinose), and 68% for the sum of sugars (mass yield in terms of EFB solid content).

The enzyme recovery rate was calculated by the following formula.

Enzyme recovery rate %=(enzyme activity of recovered liquid(reaction liquid,washing medium))/(enzyme activity loaded at beginning of saccharification reaction)

### Example 1

### (1) Pretreatment Step

In a 100-mL glass reactor, 5.5 g of EFB fiber (moisture content: 8.9%, solid content: 5.0 g) was placed, and thereto 50.0 g of a 4.0% aqueous NaOH solution as an aqueous alkali solution was added to give a mixture. The EFB was fully impregnated with the mixture (left stand under reduced pressure at room temperature for 15 minutes). The liquid-solid ratio of the mixture was 10.1 ((all the liquid components 0.5+50.0 g)/(EFB solid content 5.0 g)). Next, filtration was carried out for solid-liquid separation, and the EFB containing the aqueous alkali solution (alkali-impregnated EFB) and part of the aqueous alkali solution were separately recovered. The mass of the alkali-impregnated EFB was 16.9 g, and the liquid-solid ratio thereof after the solid-liquid separation was 2.4 ((all the liquid components 11.9 g)/(EFB solid content 5.0 g)). The mass of NaOH solid matter in the alkali-impregnated EFB was estimated to be 0.48 g (=11.9 g×0.04, or 9.5% as the ratio of alkali impregnation amount to the feedstock EFB) from the total mass of all the liquid components. The amount of the part of the aqueous alkali solution recovered by the solid-liquid separation was about 38 g. Since the recovered aqueous alkali solution was reusable, the amount of alkali used was practically 9.5%. Subsequently, the resulting alkali-impregnated EFB was placed in a 100-mL pressure-resistant reactor equipped with a thermometer and a pressure gauge, and the gas inside the reactor was replaced by nitrogen gas. The reactor was hermetically sealed and placed in an oil bath, and heat treatment was performed at 100° C. (internal temperature of the reactor) for 1 hour.

### (2) Removal Step of Removing Pretreatment-Degradation Product (Washing Step)

Subsequently, washing with water was performed. To 16.9 g of the heat-treated EFB, 50.0 g of deionized water was added. The mixture was stirred for 10 minutes to elute the solubilized biomass degradation product (pretreatment-degradation product) resulting from the pretreatment step. Solid-liquid separation was performed by filtration to give 12.8 g of pretreated EFB (wet with water) and about 53 g of filtrate (pretreated liquid, as an aqueous alkaline solution containing about 3% of the pretreatment-degradation product, pH12.7). The pretreated EFB was subjected to the subsequent saccharification step as it is without drying.

### (3) Saccharification Step

In a 50-mL glass reactor, a reaction mixture was prepared as follows.

In the reactor, 12.8 g of the pretreated EFB, 1.6 mg of tetracycline hydrochloride, 1.2 mg of cycloheximide, 20 mL of a 0.1 M acetate buffer (pH 5.5), and 0.30 g of an enzyme liquid (mixture of enzyme A and enzyme B at 1:1) were placed, and the pH of the mixture was adjusted to 5.5 with the use of a 10% aqueous acetic acid solution. Water was added thereto to make the total mass of 40.0 g. The reactor was then hermetically sealed, and saccharification was performed with shaking in a constant-temperature shaker at 45° C. for 72 hours. The saccharified liquid (reaction liquid from which an undegraded feedstock had been removed) was sampled for HPLC analysis of the sugars produced. As a result, the glucose yield was 81% (to the theoretical glucose yield), the C5-sugar yield was 83% (to the theoretical total C5-sugar yield), and the total sugar yield was 82% (to the theoretical total sugar yield), indicating that the sugar yield was high as for both glucose and C5 sugars.

### (4) Enzyme Recovery Step

The reaction mixture after the saccharification step was filtered for solid-liquid separation to give about 35 g of the saccharified liquid (containing sugars and free enzymes) and about 5 g of an undegraded residue (wet). In order to recover the enzyme remaining in the residue, 15 g of water was added to the residue, and the mixture was gently stirred for 30 minutes. Filtration was performed and the filtrate was recovered (first treated liquid, pH 5.5). Another round of washing with water was performed in the same manner to give a second treated liquid. The saccharified liquid, the first treated liquid, and the second treated liquid were combined to give a recovered liquid (about 65 g).

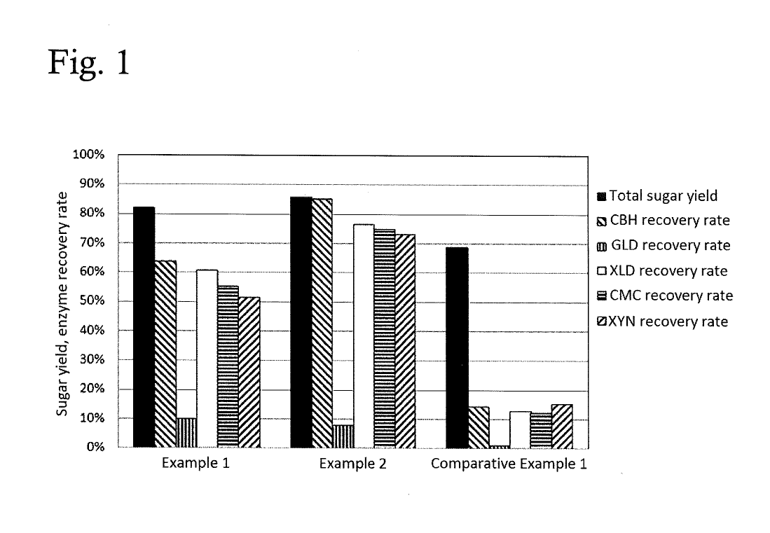

The total enzyme recovery rates in the recovered liquid were determined; 64% as for CBH, 10% as for GLD, 61% as for XLD, 55% as for CMC, and 51% as for XYN. The enzyme recovery rates of CBH, XLD, CMC, and XYN were relatively high. In spite of the known fact that GLD is extremely adsorptive and therefore hard to be recovered, the recovery rate was as high as 10%. The conditions and the results of the experiment are summarized in Table 1 and FIG. 1. The alkali amount here refers to the ratio of alkali impregnation amount in alkali-impregnated EFB (solid content basis).

### Comparative Example 1

An experiment on EFB saccharification was carried out employing the following pretreatment method with the use of NaOH in the same amount as in Example 1 but without solid-liquid separation.

That is, in a 100-mL pressure-resistant reactor equipped with a thermometer and a pressure gauge, 5.5 g of EFB fiber was placed in the same manner as in Example 1, and thereto 50.0 g of a 1.0% aqueous NaOH solution as an aqueous alkali solution was added (liquid-solid ratio: 10.1). The aqueous alkali solution contained 0.50 g of NaOH (10% to the amount of the feedstock EFB), and therefore the usage of NaOH was equivalent to that in Example 1.

After the EFB (left stand under reduced pressure at room temperature for 15 minutes) was fully impregnated with the aqueous alkali solution, without solid-liquid separation, the gas inside the reactor was replaced by nitrogen gas. The reactor was then hermetically sealed, and heat treatment and a washing step were performed in the same manner as in Example 1 to give pretreated EFB. In the washing step, however, prior to the washing process performed in the same manner, filtration was carried out to remove liquid.

A saccharification step and an enzyme recovery step (including washing with water) were performed exactly in the same manner as in Example 1, and then the sugar yield and the enzyme recovery rate were measured. The results are shown in Table 1 and FIG. 1.

### Example 2

Pretreatment was performed in the same manner as in Example 1 except that at the time of the heat treatment in the pretreatment step, the atmosphere of the gas phase was 80% by volume of oxygen/20% by volume of nitrogen instead of nitrogen. The pressure was 0.2 MPaG (gauge pressure) in terms of the total pressure at 100° C. This pressure was maintained by supplying oxygen gas so as to compensate for the pressure loss caused by the consumption of oxygen. Following the pretreatment, a washing step, a saccharification step, and an enzyme recovery step were performed in the same manner as in Example 1. The results are shown in Table 1 and FIG. 1.

### Examples 3 to 7

An experiment on EFB saccharification was performed in the same manner as in Example 1 or 2 under the conditions varied as shown in Table 1. The results are shown in Table 1. In Example 4, prior to raising the temperature, pressure was applied by introducing 80% by volume of oxygen/20% by volume of nitrogen to achieve 1.0 MPaG at room temperature. Then, heat treatment was performed without compensation for the pressure loss caused by the consumption of oxygen.

### Example 8

In this experiment, the procedure to the preparation of alkali-impregnated EFB (impregnation with 3% NaOH) in Example 5 was performed in the same manner, and then the method of heat treatment modified as below was performed. That is, the resulting alkali-impregnated EFB was placed in a 100-mL plastic beaker, the plastic beaker was placed in a 2-L plastic container, and the container was covered with a lid (at the bottom of the 2-L container, a cloth soaked with 100 mL of water was placed to make the container filled with water vapor during heat treatment). The lid had small holes so as to prevent the internal pressure from rising. The reaction container thus prepared was placed in an oven at 80° C. and left stand for 12 hours for heat treatment (the atmosphere of the gas phase was air and therefore enough oxygen was present, at atmospheric pressure). Steps after heat treatment were performed in the same manner as in Example 1. The results are shown in Table 1.

### Examples 9 and 10

An experiment on EFB saccharification was carried out in the same manner as in Example 8 under the conditions varied as shown in Table 1. The results are shown in Table 1.

### Example 11

The procedure to the preparation of alkali-impregnated EFB and the heat treatment in Example 5 was performed in the same manner. That is, heat treatment was performed in nitrogen (with limited oxygen) at 100° C. for 1 hour. Subsequently, the heat-treated EFB was subjected to another round of heat treatment with oxygen being supplied (in an air atmosphere, at atmospheric pressure) in the same manner as in Example 8 at 80° C., but this time the duration was 6 hours. Steps after heat treatment were performed in the same manner as in Example 1. The results are shown in Table 1.

### Example 12

An experiment on saccharification was carried out using EFB that was swollen with water (water-wet EFB) as a feedstock. That is, in a 100-mL pressure-resistant container, 14.6 g of water-wet EFB (moisture content: 65.8%, solid content: 5.0 g) was placed, and thereto 15.0 g of a 6.0% aqueous NaOH solution as an aqueous alkali solution was added (liquid-solid ratio=4.9; amount of all liquid components: 9.6 g+15.0 g=24.6 g; EFB solid content: 5.0 g). After gentle mixing with a stirring rod, the container was hermetically sealed and was then pressurized with air to 0.2 MPaG. The system was maintained at 40° C. for 1 hour so as to achieve impregnation of the EFB with the aqueous alkali solution. The subsequent steps were performed in the same manner as in Example 8. The results are shown in Table 2. Calculation was made to determine the NaOH concentration of all the liquid components in the alkali-impregnated EFB to be 3.7% and the alkali impregnation ratio to be 8.5%.

### Example 13

An experiment on saccharification of EFB (wet with water) was carried out in the same manner as in Example 12 except that the conditions for impregnation with an aqueous alkali solution were changed. That is, alkali impregnation was conducted under conditions at atmospheric pressure (no pressurization with air) at 70° C. for 30 minutes. The results are shown in Table 2.

### Example 14

An experiment was carried out under the same conditions for pretreatment and saccharification as in Example 1 except that the conditions for residue treatment in an enzyme recovery step were changed as follows. As the first treatment of a residue, 15 g of water was added to the residue and mixed with stirring. Thereto, a trace amount of a 1% aqueous NaOH solution was added so as to adjust the pH of the treatment liquid to 8.0. After gentle mixing for 30 minutes with stirring for enzyme desorption under alkaline conditions, filtration was performed to recover a first treated liquid. The second treatment was carried out in the same manner as in Example 1 with the use of water so as to recover a second treated liquid. The total enzyme recovery rates in the recovered liquid were measured, and the results are shown in Table 3.

### Examples 15 to 17

An experiment on EFB saccharification was carried out in the same manner as in Example 14 under the conditions for the first treatment and the second treatment of a residue in an enzyme recovery step changed as shown in Table 3. The results are shown in Table 3. In Example 15, the pH in the first treatment was 9.0. In Example 16, the pH for the first treatment was 8.0, which was increased stepwise to 9.0 during the second treatment by the addition of NaOH to the treatment liquid. Example 17 was an experiment where 10% of the pretreated liquid resulting from the washing step was added to the treatment liquid in the first treatment. The results are shown in Table 3.

### Examples 18 and 19

The conditions in the pretreatment and the saccharification were the same as in Example 2. Only the conditions for residue treatment in the enzyme recovery step were changed. The first treatment of the residue was conducted in the same manner as in Example 14 under the conditions shown in Table 3. The results are shown in Table 3.

### Example 20

In this experiment, the procedure to the EFB saccharification step was performed in the same manner as in Example 2, and then an enzyme recovery step was carried out in the same manner as in Example 16 (the pH during alkali treatment of residue: 8.0 to 9.0) to give a recovered liquid. The resulting recovered liquid as a whole was subjected to ultrafiltration (using Kurabo Centricut U-10, molecular weight cut-off: 10,000, polysulfone membrane) for concentration down to about 10 g, thereby giving a recovered enzyme liquid, which was then subjected to another round of EFB saccharification experiment (enzyme recycle reaction). That is, pretreated EFB was prepared in the same manner as in Example 2, and a reaction mixture was prepared as follows.

The pretreated EFB (wet with water), 1.6 mg of tetracycline hydrochloride, 1.2 mg of cycloheximide, 10 mL of a 0.1 M acetate buffer (pH 5.5), about 10 g of the recovered enzyme liquid, and 0.06 g of a fresh enzyme liquid (mixture of enzyme A and enzyme B at 1:1) were mixed, and the pH of the resultant was adjusted to 5.5 with a 10% aqueous acetic acid solution. Then, water was added to make the total mass of 40.0 g. The enzyme liquid (fresh) in an amount equivalent to 20% the amount of the enzyme liquid used in the first round was added for compensating for the loss.

The resultant mixture was subjected to saccharification under the same conditions as in Example 2. HPLC analysis of the resultant sugars showed that the glucose yield was 89%, the C5-sugar yield was 81%, and the total sugar yield was 86% (the sugar yields were determined taking into account the amounts of sugars derived from the recovered enzyme liquid), indicating that the sugar yields were equivalent to those in the first round.

### Example 21

An experiment on EFB saccharification was carried out under the same conditions as in Example 8 except that a different aqueous alkali solution was used in a pretreatment step. The aqueous alkali solution used was 5% KOH. The results are shown in Table 1.

### Example 22

An experiment on EFB saccharification was carried out in the same manner as in Example 1 except that, to a reaction mixture prepared in a saccharification step, 10.0 g of the pretreated liquid resulting from the washing step was added (instead, the same amount of the acetate buffer was reduced, 40.0 g in total, pH 5.5), and then saccharification was performed. The enzyme recovery step was performed in the same manner as in Example 1. The sugar yields were 83% for glucose, 85% for C5 sugars, and 84% for the sum of the sugars. The enzyme recovery rates were 71% as for CBH, 32% as for GLD, and 65% as for XLD. The sugar yields and the enzyme recovery rates were higher than those in Example 1.

### Example 23

An experiment on saccharification of rice straw was carried out in the same manner as in Example 5 except that rice straw was used as a biomass feedstock. That is, the feedstock used was 5.7 g of rice straw (produced in the prefecture of Nagano, moisture content: 11.6%, solid content: 5.0 g), which was impregnated with 3% NaOH (liquid-solid ratio: 10), and then solid-liquid separation was performed. The mass of the alkali-impregnated rice straw was 23.8 g and the liquid-solid ratio after solid-liquid separation was 3.8. The heat treatment step and later steps were performed in the same manner as in Example 5 except that the reaction time in a saccharification step was 20 hours. The sugar yields in terms of mass yields to the amount of feedstock rice straw (untreated, solid content basis) were 32% for glucose, 13% for C5 sugars, and 45% for the sum of the sugars. Provided that the theoretical sugar yield of rice straw is 70%, the obtained value was 64% thereto. The enzyme recovery rates were 95% as for CBH and 90% as for XLD, which were extremely high.

### Example 24

Alkali-impregnated EFB was prepared in the same manner as in Example 1 except that the amount of the 4% aqueous NaOH solution for impregnation was 75 g (liquid-solid ratio before solid-liquid separation: 15.1). After solid-liquid separation, the alkali-impregnated EFB was placed in a pressure-resistant reactor, of which the inside atmosphere was air. The reactor was then hermetically sealed and placed in an oil bath. The temperature was maintained at 180° C. (internal temperature of the reactor) for 15 minutes for heat treatment (heat treatment at a high temperature in a short time). A washing step and a saccharification step were performed in the same manner as in Example 1. The results are shown in Table 4, in which the composition of the saccharified liquid is also shown as the total concentration of sugars (concentration of glucose+C5 sugars) and the proportion of C5 sugars (the proportion by mass of C5 sugars to the sum of sugars).

### Examples 25 to 37

An experiment was carried out in the same manner as in Example 24 under the conditions varied as shown in Table 4, which also includes the results. Each of Examples 25 to 30 was an experiment on a different aqueous alkali solution and a different set of conditions for heat treatment. In Example 31, a mixed solution of NaOH and sodium carbonate (concentration of each: 1%) was used as an aqueous alkali solution. In Example 32, a 4% aqueous ammonia solution was used.

In Example 33, a 0.5% slurry of calcium oxide was used as an aqueous alkali solution. Impregnation was conducted under reduced pressure. Further, the reactor was hermetically sealed and then rotated in a rotator at 50° C. for 1 hour. Then, solid-liquid separation was performed to give alkali-impregnated EFB.

In Example 34, the liquid-solid ratio before solid-liquid separation was lowered. In Example 35, the liquid-solid ratio after solid-liquid separation was lowered (the liquid-solid ratio was lowered utilizing the absorption of the liquid by filter paper after the solid-liquid separation). Example 36 was an experiment on reusing an aqueous alkali solution. After alkali impregnation in Example 24, solid-liquid separation (filtration) was performed and the filtrate was reused as an aqueous alkali solution. In this case, NaOH and water were supplied to compensate for the loss so as to achieve the same composition as that in Example 24.

In Example 37, the atmosphere of the gas phase during heat treatment was 50% by volume of oxygen/50% by volume of nitrogen instead of air, and the initial pressure at the beginning of the heat treatment was 0.6 MPaG.

### Comparative Examples 2 to 4

In Comparative Example 2, the pretreatment did not include solid-liquid separation, as in Comparative Example 1. The heat treatment conditions were the same as in Example 24, that is, at 180° C. for 15 minutes. In each of Comparative Examples 3 and 4, an experiment was carried out in the same manner as in Example 24, but deionized water was used instead of the aqueous alkali solution. The conditions and the results of the experiments are shown in Table 4.

### Example 38

An experiment was carried out on reusing an enzyme adsorbed on an undegraded feedstock. That is, the procedure to the saccharification step was performed under the same conditions as in Example 25. However, at the time point of 24 hours in the saccharification step, the reaction was stopped in midstream. Filtration of the resulting reaction-mixture was performed to separate a wet undegraded feedstock (about 10 g) from a saccharified liquid (about 30 g). The undegraded feedstock was then mixed with pretreated EFB that had been prepared separately (under the same conditions as in Example 25) so as to prepare another reaction mixture (40.0 g) (the amount of enzyme added thereto, however, was 0.10 g, which was ⅓ of 0.30 g). Then, the saccharification reaction resumed at 45° C. After 72 hours, the reaction was terminated, and the saccharified liquid was analyzed. The sugar yields (to the theoretical yield, based on twice the amount of feedstock) resulting from the entire experiment were 90% for glucose, 89% for C5 sugars, and 90% for the sum of sugars.

### Example 39

The EFB pretreatment step was performed in the same manner as in Example 25. In the subsequent washing step, the same washing process with water as in Example 1 was repeated 4 times. That is, 50.0 g of deionized water was added to the heat-treated EFB and mixed for 10 minutes with stirring for elution of a pretreatment-degradation product. Then, filtration was performed for solid-liquid separation to give an EFB solid matter and filtrate (pretreated liquid 1). This washing process with water was repeated three more times to give filtrate (pretreated liquids 2 to 4) and pretreated EFB that had been thoroughly washed.

Moisture in pretreated liquids 1 to 4 was evaporated, and the amount of the solid matter (the amount of the pretreatment-degradation product) was determined. The results showed that the solid content was 1.54 g in pretreated liquid 1, 0.16 g in pretreated liquid 2, 0.03 g in pretreated liquid 3, <0.01 g in pretreated liquid 4, and 1.73 g in total. These results indicate that the amount of the pretreatment-degradation product remaining after each round of washing with water was 0.19 g (=1.73−1.54) after the first washing, 0.03 g (=0.19−0.16) after the second washing, and <0.01 g after the third washing. After the forth washing with water, the pretreated EFB was dried. The amount of the solid matter was 3.2 g. Therefore, the content of the remaining pretreatment-degradation product (=(amount of solid matter in remaining pretreatment-degradation product)/(amount of solid matter in pretreated EFB)) after each round of washing with water was estimated to be 5.9% after the first washing (with 50 g of washing water), 0.9% after the second washing (with 100 g of washing water), and <0.3% after the third washing (with 150 g of washing water). In this way, different washing methods give pretreatment feedstocks containing different concentrations of pretreatment-degradation product, and by subjecting such pretreatment feedstocks to saccharification, saccharified liquids containing different concentrations of pretreatment-degradation product can be prepared. In Example 1 (and other examples that employed the equivalent conditions), the content of the pretreatment-degradation product remaining in the pretreated EFB was about 6%, the saccharification reaction in the saccharification step was performed presumably in the presence of about 6% of the pretreatment-degradation product, and the estimated concentration of the pretreatment-degradation product in the saccharified liquid was about 7% relative to all the sugar components.

### Example 40

The influence of a pretreatment-degradation product on fermentation was investigated. An experiment on EFB saccharification was carried out in the same manner as in Example 25 except that the scale was increased 20-fold (the amount of feedstock EFB: 100 g). In the washing step, the washing process with water was repeated three times as in Example 39 (the content of the remaining pretreatment-degradation product: <0.3%). In the saccharification step, saccharification was performed without using tetracycline hydrochloride or cycloheximide. The solid content of the pretreated EFB was increased (less water was used, and the amount of the reaction mixture was 540 g). After the reaction, the reaction mixture was subjected to solid-liquid separation to give saccharified liquid A. Saccharified liquid A had a total concentration of sugars of 11.0% and a proportion of C5 sugars of 38%. This saccharified liquid was mixed with the filtrate (pretreated liquid A, containing 3.3% of pretreatment-degradation product solid content basis) obtained after the first washing with water at proportions shown in Table 5 to give saccharified liquids B to D containing the pretreatment-degradation product at different concentrations (proportions). Thus, models of saccharified liquids obtained under different washing conditions were prepared. Saccharified liquid C had the same composition as that of the saccharified liquid obtained after the first washing with water (see Example 39).

Subsequently, saccharified liquids A to D were subjected to butanol fermentation. The culture medium used had a sugar concentration of 40 g/L (in terms of the sum of sugars) after adjustment, contained a component for culturing (TYA medium) added thereto, and was adjusted to pH 6 to pH 7. As controls, an experiment where a reagent-grade glucose solution replaced the saccharified liquid of EFB (control 1) and an experiment where a reagent-grade solution of glucose and xylose (mass ratio: 6:4) replaced the same (control 2) were carried out. ATCC strain Clostridium saccharoperbutylacetonicum (ATCC 27021) was used, and after preculture, fermentation was allowed to proceed statically at 30° C. for 48 hours. The results are shown in Table 5, which includes the concentration (OD660) of cells after 33 hours and 48 hours of fermentation, the concentration of butanol produced, and the butanol mass yield (to sugars consumed). Each numerical value shows the average of two rounds of experiment.

TABLE 1

| | Pretreatment conditions | | | | | | | | | | | | | | |
|---|---|---|---|---|---|---|---|---|---|---|---|---|---|---|---|
| | | | Liquid- | Liquid- | | | | | | | | | | | |
| | | | solid | solid | | | | | | | | | | | |
| | | | ratio | ratio | | | | | | | | | | | |
| Exam- | | | before | after | | | | Sugar yield | | | | | | | |
| ple | | Solid- | solid- | solid- | | Gas | | (to theoretical yield) | | | | | | | |
| (Comp. | Aqueous | liquid | liquid | liquid | | phase | Heat | | | Sum | Total enzyme recovery rate | | | | |
| Exam- | alkali | sepa- | sepa- | sepa- | Alkali | atmo- | treatment | Glu- | C5 | of | (Total enzyme in recovered liquid) | | | | |
| ple) | solution | ration | ration | ration | amount | sphere | conditions | cose | Sugars | sugars | CBH | GLD | XLD | CMC | XYN |
| Ex. 1 | 4%NaOH | Per- | 10.1 | 2.4 | 9.5% | N₂ | 100° C., 1 h | 81% | 83% | 82% | 64% | 10% | 61% | 55% | 51% |
| | | formed | | | | | | | | | | | | | |
| Ex. 2 | 4%NaOH | Per- | 10.1 | 2.4 | 9.4% | 80% O₂ | 100° C., 1 h | 88% | 83% | 86% | 85% | 8% | 77% | 75% | 73% |
| | | formed | | | | | | | | | | | | | |
| Ex. 3 | 4%NaOH | Per- | 10.1 | 2.2 | 8.9% | 50% O₂ | 100° C., 3 h | 85% | 82% | 84% | 87% | 6% | 78% | ND | ND |
| | | formed | | | | | | | | | | | | | |
| Ex. 4 | 4%NaOH | Per- | 10.1 | 2.4 | 9.6% | 80% O₂ | 120° C., 3 h | 92% | 86% | 90% | 92% | 29% | 81% | ND | ND |
| | | formed | | | | | | | | | | | | | |
| Ex. 5 | 3%NaOH | Per- | 10.1 | 2.2 | 6.5% | N₂ | 100° C., 1 h | 79% | 88% | 82% | 62% | 5% | 56% | ND | ND |
| | | formed | | | | | | | | | | | | | |
| Ex. 6 | 2%NaOH | Per- | 10.1 | 2.3 | 4.6% | N₂ | 100° C., 1 h | 69% | 84% | 75% | 42% | 1% | 38% | ND | ND |
| | | formed | | | | | | | | | | | | | |
| Ex. 7 | 4%NaOH | Per- | 10.1 | 2.3 | 9.4% | N₂ | 150° C., 0.3 h | 83% | 79% | 81% | 66% | 11% | 66% | ND | ND |
| | | formed | | | | | | | | | | | | | |
| Ex. 8 | 3%NaOH | Per- | 10.1 | 2.3 | 6.9% | Air | 80° C., 12 h | 81% | 90% | 84% | 80% | 12% | 72% | 78% | 68% |
| | | formed | | | | | | | | | | | | | |
| Ex. 9 | 4%NaOH | Per- | 10.1 | 2.5 | 9.9% | Air | 40° C., 6 d | 80% | 84% | 81% | 78% | 12% | 71% | ND | ND |
| | | formed | | | | | | | | | | | | | |
| Ex. 10 | 6%NaOH | Per- | 10.1 | 2.0 | 11.9% | Air | 80° C., 6 h | 85% | 76% | 81% | 87% | 19% | 80% | ND | ND |
| | | formed | | | | | | | | | | | | | |
| Ex. 11 | 3%NaOH | Per- | 10.1 | 2.5 | 7.5% | Air | 100° C., 1 h + | 85% | 91% | 88% | 85% | 18% | 75% | ND | ND |
| | | formed | | | | | 80° C., 6 h | | | | | | | | |
| Ex. 21 | 5%KOH | Per- | 10.1 | 2.5 | 12.3% | Air | 80° C., 12 h | 81% | 85% | 82% | 81% | 18% | 79% | ND | ND |
| | | formed | | | | | | | | | | | | | |
| Comp. | 1%NaOH | None | 10.1 | — | 10.0% | N₂ | 100° C., 1 h | 63% | 78% | 69% | 14% | 1% | 13% | 12% | 15% |
| 1 | | | | | | | | | | | | | | | |

*The alkali amount in each example means the ratio of alkali impregnation amount in % by mass to feedstock EFB (solid content basis) calculated
based on the liquid-solid ratio after solid-liquid separation and on the concentration of the aqueous alkali solution.
*The sugar yield is in % by mass to the theoretical yield.
*The enzyme recovery rate is based on the initial enzyme activity.
*ND denotes that no measurement was performed.

TABLE 2

| | | Pretreatment conditions | | | | | | | | | | |
|---|---|---|---|---|---|---|---|---|---|---|---|---|
| | | | Liquid-solid | Liquid-solid | | | Sugar yield | | | | | |
| | | | ratio before | ratio after | Alkali | Heat | (to theoretical yield) | | | Total enzyme | | |
| | | Alkali | solid-liquid | solid-liquid | impregnation | treatment | | C5 | Sum of | recovery rate | | |
| Example | Feedstock | solution | separation | separation | conditions | conditions | Glucose | Sugars | sugars | CBH | GLD | XLD |
| Ex. 8 | Dry EFB | 3% | 10.1 | 2.3 | Room temperature, | 80° C., 12 h | 81% | 90% | 84% | 80% | 12% | 72% |
| | | NaOH | | | 15 min, | | | | | | | |
| | | | | | reduced pressure | | | | | | | |
| Ex. 12 | Water-wet | 6% | 4.9 | 2.3 | 40° C., 1 h, | 80° C., 12 h | 85% | 88% | 87% | 83% | 15% | 77% |
| | EFB | NaOH | | | pressurized | | | | | | | |
| Ex. 13 | Water-wet | 6% | 4.9 | 2.3 | 70° C., 30 min, | 80° C., 12 h | 82% | 85% | 83% | 82% | 17% | 76% |
| | EFB | NaOH | | | atmospheric | | | | | | | |
| | | | | | pressure | | | | | | | |

TABLE 3

| | Conditions for enzyme | | Total enzyme recovery | | |
|---|---|---|---|---|---|
| | recovery from residue | | rate (Total enzyme in | | |
| Ex- | | Second | recovered liquid) | | |
| ample | First treatment | treatment | CBH | GLD | XLD |
| Ex. 1 | Water | Water | 64% | 10% | 61% |
| Ex. 14 | NaOH added (pH 8.0) | Water | 75% | 41% | 72% |
| Ex. 15 | NaOH added (pH 9.0) | Water | 76% | 50% | 73% |
| Ex. 16 | NaOH added (pH 8.0) | NaOH added | 78% | 65% | 75% |
| | | (pH 9.0) | | | |
| Ex. 17 | Pretreated liquid 10% + | Water | 76% | 52% | 74% |
| | NaOH added (pH 8.0) | | | | |
| Ex. 2 | Water | Water | 85% | 8% | 77% |
| Ex. 18 | NaOH added (pH 8.0) | Water | 93% | 62% | 84% |
| Ex. 19 | NaOH added (pH 9.0) | Water | 94% | 78% | 85% |

TABLE 4

| | | | | | | | | | | Composition of | |
|---|---|---|---|---|---|---|---|---|---|---|---|
| | Pretreatment conditions | | | | | | Sugar yield | | | saccharified liquid | |
| | | | Liquid-solid | Liquid-solid | | | (to theoretical yield) | | | Total | |
| Example | Aqueous | Solid- | ratio before | ratio after | | | | | Sum | concen- | |
| (Comp. | alkali | liquid | solid-liquid | solid-liquid | Alkali | Heat treatment | | C5 | of | tration | C5 sugar |
| Example) | solution | separation | separation | separation | amount | conditions | Glucose | Sugars | sugars | of sugars | proportion |
| Ex. 24 | 4%NaOH | Performed | 15.1 | 2.3 | 9.0% | 180° C., 15 min | 95% | 81% | 90% | 7.6% | 35% |
| Ex. 25 | 3%NaOH | Performed | 15.1 | 2.3 | 6.8% | 180° C., 15 min | 93% | 90% | 92% | 7.8% | 37% |
| Ex. 26 | 2%NaOH | Performed | 15.1 | 2.3 | 4.6% | 180° C., 15 min | 91% | 92% | 91% | 7.7% | 38% |
| Ex. 27 | 1%NaOH | Performed | 15.1 | 2.3 | 2.3% | 180° C., 15 min | 70% | 80% | 74% | 6.2% | 42% |
| Ex. 28 | 3%NaOH | Performed | 15.1 | 2.3 | 6.8% | 120° C., 15 min | 80% | 96% | 86% | 7.3% | 43% |
| Ex. 29 | 3%NaOH | Performed | 15.1 | 2.3 | 6.9% | 150° C., 15 min | 83% | 94% | 87% | 7.4% | 41% |
| Ex. 30 | 3%NaOH | Performed | 15.1 | 2.3 | 6.8% | 200° C., 15 min | 91% | 69% | 83% | 7.0% | 32% |
| Ex. 31 | 1%NaOH + | Performed | 15.1 | 2.3 | 4.5% | 190° C., 15 min | 76% | 68% | 73% | 6.2% | 36% |
| | 1%Na₂CO₃ | | | | | | | | | | |
| Ex. 32 | 4%NH₃ | Performed | 15.1 | 2.4 | 9.6% | 180° C., 15 min | 70% | 68% | 69% | 5.4% | 35% |
| Ex. 33 | 0.5%CaO | Performed | 15.1 | 2.3 | — | 200° C., 15 min | 94% | 38% | 72% | 6.1% | 20% |
| Ex. 34 | 3%NaOH | Performed | 8.0 | 2.2 | 6.7% | 180° C., 15 min | 91% | 90% | 91% | 7.7% | 38% |
| Ex. 35 | 3%NaOH | Performed | 5.0 | 1.5 | 4.5% | 180° C., 15 min | 85% | 88% | 86% | 7.3% | 39% |
| Ex. 36 | 3%NaOH | Performed | 15.1 | 2.3 | 6.8% | 180° C., 15 min | 94% | 89% | 92% | 7.8% | 37% |
| | (reused) | | | | | | | | | | |
| Ex. 37 | 3%NaOH | Performed | 15.1 | 2.3 | 6.9% | 150° C., 15 min | 90% | 86% | 88% | 7.5% | 37% |
| | | | | | | 50% oxygen | | | | | |
| | | | | | | atmosphere | | | | | |
| Comp. Ex. 2 | 1%NaOH | None | 10.1 | — | 10.0% | 180° C., 15 min | 68% | 65% | 67% | 5.7% | 37% |
| Comp. Ex. 3 | H₂O | Performed | 15.1 | 2.3 | 0% | 180° C., 15 min | 71% | 32% | 56% | 4.8% | 22% |
| Comp. Ex. 4 | H₂O | Performed | 15.1 | 2.3 | 0% | 210° C., 15 min | 90% | 3% | 57% | 4.8% | 2% |

TABLE 5

| | | | Composition | | | | | | | | |
|---|---|---|---|---|---|---|---|---|---|---|---|
| | | | | | Concentration | | | | | | |
| | | | | Concentration | of | Fermentation results | | | Fermentation results | | |
| | | | | of | pretreatment- | (33-h fermentation) | | | (48-h fermentation) | | |
| | Mixing ratio | | Sugar | pretreatment- | degradation | | Butanol | | | Butanol | |
| Saccharified | Saccharified | Pretreated | concen- | degradation | product/sugar | | concen- | To sugar | | concen- | To sugar |
| liquid | liquid A | liquid | tration | product | concentration | OD660 | tration | yield | OD660 | tration | yield |
| A | 1 | 0 | 11.0% | 0% | 0% | 8.6 | 0.89% | 29.4% | 9.0 | 1.22% | 29.1% |
| B | 1 | 0.05 | 10.5% | 0.16% | 2% | 9.9 | 1.05% | 32.0% | 8.7 | 1.33% | 31.8% |
| C | 1 | 0.15 | 9.6% | 0.43% | 5% | 8.5 | 0.90% | 32.1% | 8.7 | 1.23% | 31.0% |
| D | 1 | 0.4 | 7.9% | 0.94% | 12% | 5.3 | 0.48% | 30.3% | 8.6 | 1.06% | 31.0% |

| | | | | | | | | | |
|---|---|---|---|---|---|---|---|---|---|
| Control 1 | — | — | Reagent-grade glucose | 7.0 | 0.70% | 30.1% | 10.0 | 0.99% | 28.8% |
| Control 2 | — | — | Reagent-grade glucose + xylose (6:4) | 6.9 | 0.69% | 29.6% | 9.7 | 0.95% | 29.0% |

* The concentration of sugar and the concentration of pretreatment-degradation product are based on solid content.

As shown in Table 1, Example 1, where solid-liquid separation was performed after alkali impregnation, gave a 13% higher total sugar yield than that in Comparative Example 1 not involving solid-liquid separation, even though less alkali was used in Example 1. From the fact that the enzyme recovery rate was high in Example 1 but extremely low in Comparative Example 1, the present invention was proven to be superior also in enzyme recovery. The liquid-solid ratio in the pretreatment in Example 1 was 2.4 while that in Comparative Example 1 was 10. The water usage in Example 1 was about a quarter, which means that the water usage can be reduced.

Example 2 confirmed that the addition of oxygen during the pretreatment further increased the sugar yield and the enzyme recovery rate. Example 3 showed that a low oxygen concentration still resulted in a high sugar yield and a high enzyme recovery rate. Example 4 showed that changes in pressure and heat treatment conditions further increased the sugar yield and the enzyme recovery rate. In particular, the GLD recovery rate was improved. Examples 5 and 6, each of which was an experiment on a lower alkali concentration (less alkali) for impregnation, gave a sugar yield and an enzyme recovery rate that were higher than those in Comparative Example 1, showing that the usage of alkali can be reduced. Example 7, where heat treatment was performed at a high temperature in a short time, gave a high sugar yield and a high enzyme recovery rate. Examples 8 to 10 showed that heat treatment at normal pressure under an air atmosphere with a low oxygen concentration at a low temperature for a prolonged period of time still gave a high sugar yield and a high enzyme recovery rate. Example 11, where heat treatment was performed in 2 steps (with no oxygen supplied+with oxygen supplied), gave a high sugar yield and a high enzyme recovery rate in less time. Example 21, where an alkali other than NaOH was used, gave excellent results.

As shown in Table 2, each of Examples 12 and 13 was an experiment on a feedstock wet with water (high in the moisture content) carried out under different conditions for alkali impregnation, and showed that such conditions also gave a high sugar yield and a high enzyme recovery rate.

Table 3 shows the results of enzyme recovery from a residue under different conditions. Examples 14 and 15 confirmed that the addition of alkali in the enzyme recovery step improved the enzyme recovery rate, indicating that the recovery rate rises as the pH increases. Example 16, where the pH during alkali treatment was progressively increased, gave a higher enzyme recovery rate. Example 17 showed that adding a pretreatment-degradation product in the enzyme recovery process gave a higher enzyme recovery rate than that in Example 14, where the same pH conditions were employed. Examples 18 and 19 showed that the presence of oxygen during the pretreatment and the addition of alkali in the enzyme recovery process gave a higher enzyme recovery rate. In particular, the GLD recovery rate was improved.

Example 20 was an experiment where a recovered enzyme was reused to give results equivalent to ones from the first round of reaction, and therefore showed that the recovered enzyme was reusable. Example 22 showed that adding a pretreatment-degradation product in a saccharification reaction increased the sugar yield and the enzyme recovery rate. Example 23 was an experiment on rice straw, which was herbaceous biomass, used as the feedstock and showed that the use of rice straw also gave a high sugar yield and a high enzyme recovery rate.

Examples 24 to 37 showed that heat treatment in the pretreatment step performed at a high temperature (about 150° C. to 200° C.) in a short time gave a high sugar yield. The sugar concentration and the proportion of C5 sugars are shown as well. Example 36 showed that an aqueous alkali solution recovered in solid-liquid separation was reusable. Comparison with Comparative Examples 2 to 4 confirmed that solid-liquid separation and the use of alkali increased the sugar yield even at high temperatures.

Example 38 showed that the sequential saccharification reaction performed by reusing an enzyme adsorbed on biomass effectively reduced the enzyme usage and enhanced sugar production. Example 39, where the removal step of removing the pretreatment-degradation product was examined, clarified how the washing method affected the content of the remaining pretreatment-degradation product. Example 40 was an experiment on fermentation of a saccharified liquid and gave excellent fermentation results. It was also found that the presence of the pretreatment-degradation product in the saccharified liquid increased the concentration of the fermentation product or the sugar yield.

Thus, the method of the present invention, where an alkali efficiently acts on biomass, can give a high sugar yield and a high enzyme recovery rate as well with less alkali and water. The presence of oxygen in the pretreatment can further increase the sugar yield and the enzyme recovery rate. The method of the present invention can also give a saccharified liquid with excellent fermentation properties while reducing the load of the removal of a pretreatment-degradation product.

In addition to the embodiments and examples described above, various modifications can be made to the present invention within the scope explained in the present specification. The technical scope of the present invention also includes embodiments where technical means disclosed in separate embodiments are combined as needed.

### INDUSTRIAL APPLICABILITY

The present invention provides a useful method for saccharification of lignocellulosic biomass to give sugars for use as a fermentation feedstock.

1. A method for saccharification of lignocellulosic biomass, the method comprising (1) a pretreatment step of impregnating lignocellulosic biomass with an aqueous alkali solution, subjecting the resultant mixture to solid-liquid separation to remove part of the aqueous alkali solution, and then performing heat treatment, and (2) a saccharification step of enzymatically degrading the lignocellulosic biomass resulting from the pretreatment step to obtain a saccharified liquid.

2. The saccharification method according to claim 1, wherein the liquid-solid ratio of the mixture as calculated by Formula (I) is 2 to 20 before solid-liquid separation and 1 to 6 after solid-liquid separation in the pretreatment step.
Liquid-solid ratio=(total mass of all liquid components in mixture)/(mass of solid matter of lignocellulosic biomass in mixture) Formula (I)

3. The saccharification method according to claim 1, wherein the heat treatment in the pretreatment step is performed at 100 to 200° C.

4. The saccharification method according to claim 1, wherein the saccharification step is performed in the presence of solubilized lignocellulosic biomass that is a pretreatment-degradation product resulting from the pretreatment step.

5. The saccharification method according to claim 1, the method further comprising, between the pretreatment step and the saccharification step, a removal step of partially removing a solubilized lignocellulosic biomass that is a pretreatment-degradation product resulting from the pretreatment step, wherein the content of the pretreatment-degradation product remaining in the lignocellulosic biomass after the removal step is 2 to 20% by mass as calculated by Formula (II).
Content of remaining pretreatment-degradation product=(mass of solid matter of remaining pretreatment-degradation product)/(mass of solid matter of lignocellulosic biomass) Formula (II)

6. The saccharification method according to claim 1, wherein the proportion of C5 sugar to all the sugar components in the saccharified liquid obtained in the saccharification step is 20 to 50% by mass.

7. The saccharification method according to claim 1, wherein the total sugar concentration of the saccharified liquid resulting from the saccharification step is 5 to 20% by mass.

8. The saccharification method according to claim 1, wherein an enzyme adsorbed on the lignocellulosic biomass that remains undegraded in the saccharification step is reused.

9. The saccharification method according to claim 1, wherein the heat treatment in the pretreatment step is performed with the supply of oxygen.

10. The saccharification method according to claim 1, the method further comprising, after the saccharification step, an enzyme recovery step of recovering an enzyme after the completion of the saccharification step.

11. The saccharification method according to claim 10, wherein the enzyme recovery step includes a step of desorbing and recovering the enzyme adsorbed on the undegraded lignocellulosic biomass by alkali treatment.

12. The saccharification method according to claim 1, wherein lignocellulosic biomass with a moisture content of 30 to 90% is subjected to the pretreatment step.

13. A saccharified liquid resulting from a saccharification step, the saccharified liquid comprising 2 to 20% by mass of solubilized lignocellulosic biomass that is a pretreatment-degradation product resulting from a pretreatment step, relative to all the sugar components in the saccharified liquid.

14. The saccharification method according to claim 2, wherein the heat treatment in the pretreatment step is performed at 100 to 200° C.

15. The saccharification method according to claim 2, wherein the saccharification step is performed in the presence of solubilized lignocellulosic biomass that is a pretreatment-degradation product resulting from the pretreatment step.

16. The saccharification method according to claim 3, wherein the saccharification step is performed in the presence of solubilized lignocellulosic biomass that is a pretreatment-degradation product resulting from the pretreatment step.

17. The saccharification method according to claim 2, the method further comprising, between the pretreatment step and the saccharification step, a removal step of partially removing a solubilized lignocellulosic biomass that is a pretreatment-degradation product resulting from the pretreatment step, wherein the content of the pretreatment-degradation product remaining in the lignocellulosic biomass after the removal step is 2 to 20% by mass as calculated by Formula (II).
Content of remaining pretreatment-degradation product=(mass of solid matter of remaining pretreatment-degradation product)/(mass of solid matter of lignocellulosic biomass) Formula (II)

18. The saccharification method according to claim 3, the method further comprising, between the pretreatment step and the saccharification step, a removal step of partially removing a solubilized lignocellulosic biomass that is a pretreatment-degradation product resulting from the pretreatment step, wherein the content of the pretreatment-degradation product remaining in the lignocellulosic biomass after the removal step is 2 to 20% by mass as calculated by Formula (II).
Content of remaining pretreatment-degradation product=(mass of solid matter of remaining pretreatment-degradation product)/(mass of solid matter of lignocellulosic biomass) Formula (II)

19. The saccharification method according to claim 4, the method further comprising, between the pretreatment step and the saccharification step, a removal step of partially removing a solubilized lignocellulosic biomass that is a pretreatment-degradation product resulting from the pretreatment step, wherein the content of the pretreatment-degradation product remaining in the lignocellulosic biomass after the removal step is 2 to 20% by mass as calculated by Formula (II).
Content of remaining pretreatment-degradation product=(mass of solid matter of remaining pretreatment-degradation product)/(mass of solid matter of lignocellulosic biomass) Formula (II)

20. The saccharification method according to claim 2, wherein the proportion of C5 sugar to all the sugar components in the saccharified liquid obtained in the saccharification step is 20 to 50% by mass.
